(19) United States
(12) Patent Application Publication
(10) Pub. No.: US 2003/0220535 A1
(43) Pub. Date: Nov. 27, 2003

(54) Device for retaining material

(76) Inventor: Patrick J. Ferguson, Portland, OR (US)

Correspondence Address: Glenn C.Brown, P.C., Suite 308, 777 N.W. Wall Street, Bent, OR, 97701, US

(21) Appl. No.: 10/407,803

(22) Filed: Apr. 4, 2003

Related U.S. Application Data
(60) Provisional application No. 60/370,546, filed on Apr. 4, 2002.

(51) Int. Cl. A61N 5/00

(52) U.S. Cl. 600/8

(57) ABSTRACT

A stopper for holding one part of a surgical needle assembly approximately fixed relative to another part includes a planar rectangular body having two opposing faces, and a protrusion that extends away from the body. Each face typically includes a central depression configured for gripping by a finger or thumb. A needle assembly, for use with the stopper of the present invention, typically includes a hollow needle with a gripping member mounted on its proximal end, and a push rod configured to slidingly engage the hollow needle. The stopper is typically configured to fit snugly between the gripping member and the push rod to prevent movement of the push rod relative to the hollow needle during use.

### CROSS-REFERENCE TO RELATED APPLICATIONS

This application claims priority to U.S. Provisional Patent Application Serial No. 60/370,546, filed Apr. 4, 2002 and entitled “A Device for Retaining Material”, which is hereby incorporated by reference.

### TECHNICAL FIELD

The present invention relates generally to surgical tools and, more specifically to a stopper useful for holding one part of a surgical needle assembly approximately fixed relative to another part.

### BACKGROUND OF THE INVENTION

Hollow needle/push rod assemblies can be used for a variety of medical applications and procedures. Typically the push rod fits within, and is free to move along the longitudinal axis of the hollow needle. Such hollow needle/push rod assemblies often are used when depositing, sampling or removing material within living tissue.

The detailed description of the invention, below, is based on an embodiment of the present invention that may be used in prostate brachytherapy, in which radioactive seeds are implanted in a human prostate while the needle and seeds are monitored by ultrasound sensors and imaging devices. U.S. Pat. Nos. 4,461,280, 4,700,692 and 4,815,449, the disclosures of which are incorporated herein by reference, disclose background information about the deposition of radioactive seeds within human tissue.

As discussed below, manipulation of loaded hollow needle/push rod assemblies can be difficult, requiring a user to perform several assembly-related tasks simultaneously. During assembly preparation, the radioactive seeds are loaded into the cavity of the hollow needle until the first loaded seed abuts the distal end of the push rod. By retracting the push rod to a pre-defined position, relative to the needle, a user can load the desired number of seeds, or seeds disposed between spacers. Loaded or unloaded, the push rod is typically free to move within the needle assembly, as dictated by gravitational forces. Thus, to avoid unwanted seed ejection of a loaded assembly, the push rod typically must be manually held in a fixed position (relative to the needle). A user is therefore burdened not only with performing sensitive assembly manipulation during seed implantation, but also with holding the push rod and needle in a fixed orientation so that the seeds don't inadvertently fall out.

### SUMMARY OF THE INVENTION

A stopper is provided that is useful for holding one part of a surgical needle assembly approximately fixed relative to another part. The stopper typically includes a planar rectangular body having two opposing faces, and a protrusion that extends away from the body. Each face typically includes a central depression configured for gripping by a finger or thumb. A needle assembly, for use with the stopper of the present invention, typically includes a hollow needle with a gripping member mounted on its proximal end, and a push rod configured to slidingly engage the hollow needle. The stopper is typically configured to fit snugly between the gripping member and the push rod to prevent movement of the push rod relative to the hollow needle during use.

### BRIEF DESCRIPTION OF THE DRAWINGS

FIG. 1 is a photograph of a stopper, according to one embodiment of the present invention.

FIG. 2 is a perspective view of a stopper, according to another embodiment of the present invention.

FIG. 3 is a photograph showing a surgical needle assembly for use with the stopper shown in FIG. 1.

FIG. 4 is a photograph of the surgical needle assembly shown in FIG. 3 showing the push rod partially retracted from the hollow needle.

FIG. 5 is a photograph of the assembly shown in FIG. 4 including the stopper of FIG. 1.

### DETAILED DESCRIPTION OF THE PREFERRED EMBODIMENTS

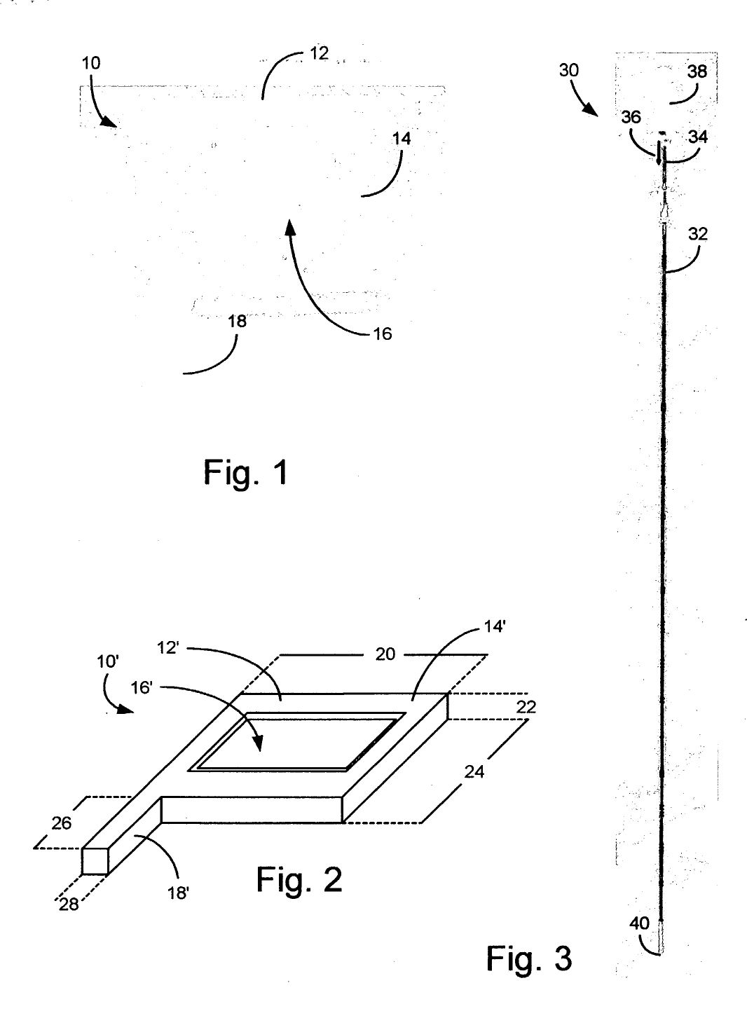

Referring initially to FIG. 1, a stopper for use with a surgical needle assembly, according to one embodiment of the present invention in generally shown at 10. Stopper 10 typically includes a substantially planar rectangular body 12 having two opposing faces, one face being shown generally at 14. Each face typically includes depression 16 configured to facilitate gripping of stopper 10 by a user. In an alternative form of the present invention, planar body 12 may contain a cylindrical opening, located at its center. Stopper 10 also typically includes protrusion 18 that is configured to hold one part of a surgical needle assembly approximately fixed relative to another part. Typically body 12 has rounded edges and protrusion 18 is cylindrical in shape, narrowing slightly as it extends away from body 12.

Referring next to FIG. 2, a perspective view of a stopper is shown according to one embodiment of the present invention generally at 10′. Typically, rectangular body 12′ is approximately 16 to 20 mm wide, 2 to 4 mm thick, and 12 to 16 mm long, as shown respectively at 20, 22, and 24. In a preferred embodiment, body 12′ is 18 mm wide, 3 mm thick, and 15 mm long. Protrusion 18′ typically extends from and is parallel to planar body 12′. In a preferred embodiment of the present invention, protrusion 18′ is flush with one side of body 12′. Alternatively, protrusion 18′ may be oriented in any other configuration, with respect to body 12′, that allows stopper 10′ to hold a push rod in a fixed position relative a hollow needle, as described above. Protrusion 18′ is typically approximately 4 to 6 mm long and 1 to 2 mm wide as shown at 26 and 28, respectively. In a preferred embodiment protrusion 18′ is 5 mm long and 1.3 mm wide.

Referring next to FIG. 3, a surgical needle assembly for use with stoppers 10, 10′ is shown generally at 30. Assembly 30 typically includes hollow needle 32 and push rod 34. Needle 32 is typically cylindrical in shape, its length being substantially greater than its diameter. Hollow gripping member 36 is typically mounted on the proximal end of needle 32. Member 36 is typically conical in shape, is composed of a rigid plastic material, and is configured such that it can be easily gripped by a finger and/or thumb. Push rod 34 typically fits within the hollow cavity of needle 32 and is configured to slide within needle 32 to deposit material stored therein. Alternatively, push rod 34 can be retracted from the needle cavity so that needle 32 can be loaded with material. Typically, push rod 34 does not engage gripping member 36. However, handling element 38, mounted on one end of push rod 34 is configured to engage member 36 as push rod 34 slides toward needle tip 40 of hollow needle 32. When gripping member 36 and handling element 38 are engaged, push rod 34 typically extends completely through the cavity of needle 32 to deposit loaded material. The material deposited is typically radioactive seeds or radioactive seeds disposed between spacers, useful for treating tumors.

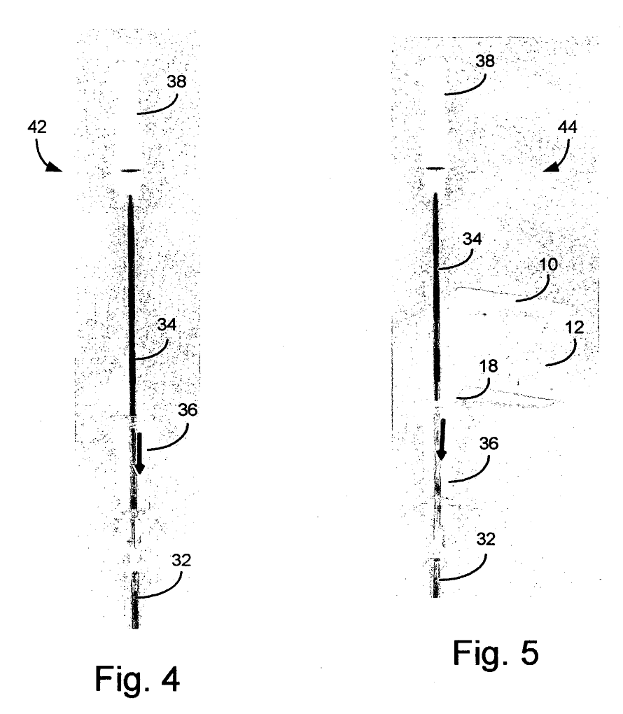

Referring next to FIG. 4, the assembly of FIG. 3 is shown with the push rod partially retracted from the hollow needle generally at 42. In most orientations, gravitational forces cause push rod 34 to either fall out of hollow needle 32 or slide towards tip 40 (not shown here) until handling element 38 engages gripping member 36, as described above. Holding push rod 34 stationary at a fixed position, relative to needle 32, is typically accomplished by manually holding push rod 34, needle 32, or both. This creates an additional burden on a user who must manipulate the assembly for seed implantation while simultaneously maintaining push rod 34 in its fixed orientation.

Referring next to FIG. 5, the assembly of FIG. 4 is shown including a stopper according to the embodiment shown in FIG. 1, generally at 44. Typically, protrusion 18 of stopper 10 fits snugly between gripping member 36 and push rod 34, substantially limiting push rod 34's movement with respect to needle 32. Typically, stopper 10 is configured to hold push rod 34 in any fixed position, relative to needle 32, as long as the push rod is at least partially within the needle cavity. Maintaining push rod 34 in a fixed position reduces the tasks required for use of hollow needle/push rod assembly 30 and other similar types of assemblies. Thus, the user is free to concentrate on assembly manipulation, which improves the precision and consistency of seed implantation during brachytherapy.

Although the invention has been disclosed in its preferred forms, the specific embodiments thereof as disclosed and illustrated herein are not to be considered in a limiting sense, because numerous variations are possible. The subject matter of the invention includes all novel and non-obvious combinations and subcombinations of the various elements, features, functions, and/or properties disclosed herein.

I claim:

1. A stopper for preventing movement of push rod of a surgical needle assembly that also includes a needle with a gripping member mounted on its proximal end, comprising:
a planar rectangular body having two opposing faces, and constructed to fit snugly between the gripping member and the push rod to prevent movement of the push rod relative to the hollow needle during use.
